(19) United States
(12) Patent Application Publication
(10) Pub. No.: US 2018/0369174 A1
(43) Pub. Date: Dec. 27, 2018

(54) METHODS AND COMPOSITIONS FOR TREATING PERIPHERAL NEUROPATHY

(71) Applicant: Achelios Therapeutics, Inc., Chapel Hill, NC (US)

(72) Inventors: Crist J. FRANGAKIS, Chapel Hill, NC (US); William WILKISON, Raleigh, NC (US); Servet BUYUKTIMKIN, San Diego, CA (US); Nadir BUYUKTIMKIN, San Diego, CA (US); James L. YEAGER, Lake Forest, IL (US)

(21) Appl. No.: 16/062,353

(22) PCT Filed: Dec. 16, 2016
(86) PCT No.: PCT/US16/67276

§ 371 Date: Jun. 14, 2018

Related U.S. Application Data
(60) Provisional application No. 62/268,007, filed on Dec. 16, 2015.

(51) Int. Cl. A61K 31/192; A61K 47/10; A61K 47/14; A61K 9/12; A61K 31/196; A61K 45/06; A61K 9/00; A61P 25/02

(52) U.S. Cl. CPC A61K 31/192; A61K 47/10; A61K 47/14; A61K 9/12; A61K 2300/00; A61K 45/06; A61K 9/0014; A61P 25/02; A61K 31/196

(57) ABSTRACT

The present disclosure provides methods and compositions which treat, alleviate, prevent, diminish or otherwise ameliorate the symptoms associated with peripheral neuropathies (e.g., neuropathic pain) or sunburn in a subject. Specifically, the present invention relates to topically administering a therapeutically effective amount of a pharmaceutically safe therapeutic agent directly to the region exhibiting pain in a subject.

### FIELD OF THE INVENTION

This invention relates to compositions for treating pain and methods of medical use.

### BACKGROUND OF THE INVENTION

Pain is an unpleasant sensory and emotional experience associated with actual or potential tissue damage or described in such terms. In considering approaches to the treatment of pain, it is important to understand the distinction between acute and persistent or chronic pain. Acute pain occurs as a result of tissue injury and is mediated by chemical, mechanical, or thermal stimulation of pain receptors known as nociceptors. In contrast to acute pain, chronic or persistent pain in itself constitutes a disease which serves no protective biological function. Chronic pain is unrelenting and can persist for years and frequently cannot be associated with a single injury. Chronic pain predominantly constitutes chronic inflammatory pain (e.g. arthritis) or “neuropathic pain” which can be defined as pain initiated or caused by a primary lesion or dysfunction within the nervous system (Mersky and Bogduk Classifications of Chronic Pain, 2.sup.nd edn. Seattle IASP Press: 394, 1994, De Andres and Garcia-Ribas Pain Practice 3:1-7, 2003). Neuropathic pain is associated with a variety of disease states and presents in the clinic with a wide range of symptoms (Woolf and Mannion Lancet 353:1959-64, 1999). It does not require specific pain receptor stimulation although such stimulation can add to the intensity of the pain sensation (Baron Clin J Pan 16 (suppl2):S12-S20, 2003).

Neuropathic pain may result from disorders of the peripheral nervous system (e.g., peripheral neuropathies) or the central nervous system (i.e., brain and spinal cord). Peripheral neuropathies are classified by the number or type of nerves affected or the process affecting the nerves. The main classifications of peripheral neuropathy are mononeuropathy, mononeuritis multiplex, polyneuropathy, autonomic neuropathy, or neuritis. Peripheral neuropathies can be caused by many factors including genetic diseases, metabolic or endocrine disorders (e.g., diabetes), exposure to toxins (e.g., drugs, heavy metals), inflammatory diseases (e.g., systemic lupus erythematosis), vitamin deficiency (e.g., vitamin B₁₂, vitamin A), physical trauma, certain infectious diseases (e.g., HIV, herpes simplex), cancer, radiation, and chemotherapy.

Neuropathic pain can be characterized by the following clinical features (Teng and Mekhail Pain Practice 3:8-12, 2003, Rajbhandari et al Pain, 83:627-629, 1999, Melzack et al Ann NY Acad Sci, 933: 157-174, 2001):
1. The presence of an abnormal, unpleasant sensation (dysesthesia) that frequently has a burning or electrical quality with an occasional paroxysmal, brief, shooting, or stabbing quality.
2. Although the onset of most neuropathic pain is within days after the precipitating injury, there is no absolute temporal relationship to the originating neural trauma such that it can begin weeks, months, or even years later.
3. Pain may be felt in a region of sensory deficit.
4. Non-noxious stimuli may be painful (allodynia).
5. Noxious stimuli may produce greater than normal response (hyperalgesia).
6. There may be an increase in the intensity of pain with repeated stimuli and the pain may persist after the removal of stimuli.

Treatment with certain chemotherapeutic agents, such as vincristine, taxol or oxaliplatin, causes in most cases the development of dose-limiting chronic neurotoxicity. The toxic damage is made evident by ensuing neural dysfunctions such as mechanical and cold allodynia, ongoing burning pain, myalgias, tingling, and numbness (Cavaliere R. and Schiff D. 2006, Curr. Neurol. Neurosci. Rep. 6:218-26). The resulting pathological condition is also known as chemotherapy-induced peripheral neuropathy (CIPN). CIPN represents an important cause of discomfort and suffering in patients undergoing chemotherapy, which strongly limits the practical applicability of the latter. In patients with CIPN symptoms, the interruption of chemotherapy is not a valid solution since it leads to worsening of the underlying disease while neurotoxicity is not necessarily removed, as it may persist or even worsen after discontinuation of medication. The degree of severity of CIPN depends not only on the drug, duration, and dose used but also on the total cumulative dose applied.

Currently, CIPN is generally alleviated by dose reduction, which may compromise the efficacy of the chemotherapy treatment. Patients who already have neuropathic symptoms due to diabetes mellitus, hereditary neuropathies, or earlier treatment with neurotoxic chemotherapy are thought to be more vulnerable to the development of CIPN.

As there are limited options currently available for the treatment of chemotherapy-induced neurotoxicity, the need is thus felt for effective therapies in the treatment of CIPN. The need is felt for agents capable of improving the tolerability of chemotherapeutic agents, thereby increasing the clinical acceptance of the latter. A further need is that of suitable co-therapies capable of positively synergizing in the effective treatment of cancer, whereby the neurotoxic side-effects of chemotherapeutic agents are inhibited. It is also desired to identify pharmaceutical compositions suitable for co-therapy, whereby one agent inhibits the neurotoxic effect of the chemotherapeutic agent and a second agent blocks the neurotoxic effects of CIPN that can develop after discontinuation of the chemotherapeutic agent.

### SUMMARY OF THE INVENTION

The present invention provides methods and compositions which treat, alleviate, prevent, diminish or otherwise ameliorate the symptoms associated with peripheral neuropathies (e.g., neuropathic pain) in a subject. Reference to “neuropathic pain” includes the neuropathic component of nociceptive pain. In particular, the present invention contemplates a method for inducing an analgesic response to neuropathic pain in mammals, particularly humans, by using a novel method to topically administer anti-inflammatory agents. Specifically, the invention provides an improvement to current methods for treating and preventing the symptoms of peripheral neuropathies including neuropathic pain. More specifically, the present invention relates to topically administering a therapeutically effective amount of a pharmaceutically safe therapeutic agent directly to the region exhibiting pain in a subject. In a related aspect, the compositions and methods of the present invention do not induce overt sedation and/or reduce the side effects associated with agents used in the treatment of pain.

Accordingly, in a first aspect the invention features a method for treating peripheral neuropathy or treating pain or a symptom associated with sunburn. This method includes administering to a subject in need thereof an effective amount of a topical composition including: a) a non-steroidal anti-inflammatory drug (NSAID), e.g., ketoprofen, in the amount of 1 to 15 percent (e.g., 1% to 5%, 4% to 9%, 8% to 12%, 11% to 15%) based on the weight of the composition; b) a non-basic polymeric skin penetration enhancer (e.g., a cross-linked polyacrylic acid interpolymer, a cross-linked polyacrylic acid homopolymer, or a mixture thereof) present in an amount sufficient to enhance skin penetration (e.g., 1.5 to 1.75 percent based on the weight of the composition) of the NSAID, the polymeric skin penetration enhancer being selected from a water-dispersable acid polymer, a polysaccharide gum, or a mixture thereof; and a carrier consisting essentially of water; c) a lipophilic solvent which is a mixture of one or more aliphatic C₂ to C₈ alcohols and an aliphatic C₈ to C₃₀ ester (e.g., a mixture of ethyl alcohol, isopropyl alcohol, and isopropyl myristate), the lipophilic solvent being present in an amount of about 10 percent to about 40 percent (e.g., 10% to 15%, 13% to 18%, 16% to 20%, 18% to 25%, 22% to 28%, 25% to 30%, 28% to 33%, 32% to 35%, 33% to 40%), wherein the ratio of the amount by weight of said aliphatic alcohol to the amount by weight of said ester is in the range of about 1 to about 7. In some embodiments, the composition does not include an anesthetic (e.g., amethocaine, cocaine, prilocaine, bupivacaine, levobupivacaine, ropivacaine, mepivacaine, dibucaine, benzocaine, physostigmine, neostigmine, lidocaine, novocaine or procaine). In other embodiments, the composition does not include benzyl alcohol.

In some embodiments, the composition of the method further includes d) a chelating agent (e.g., the disodium salt of ethylenediamineteraacetic acid) in the amount of 0.01 to 0.1 percent (e.g., 0.01 to 0.03, 0.02 to 0.04, 0.03 to 0.05, 0.04 to 0.06, 0.05 to 0.07, 0.06 to 0.08, 0.07 to 0.09, or 0.08 to 0.10) based on the weight of the composition.

In other embodiments, any of the foregoing compositions of the method further include e) oxybenzone in the amount of 1 to 15 percent (e.g., 1% to 4%, 3% to 5%, 4%, to 6%, 5% to 7%, 6% to 9%, 8% to 10%, 9% to 11%, 10% to 12%, 11% to 13%, 12% to 14%, 13% to 15%) based on the weight of the composition. Preferably, when the active is oxybenzone, the concentration is less than 10% (w/w) (e.g., 1% to 9%, 1% to 8%, 1% to 7%, 1% to 6%, 1% to 5%, 2% to 9%, 2% to 8%, 2% to 7%, 2% to 6%, 2% to 5%, 3% to 9%, 3% to 8%, 3% to 7%, 3% to 6%, or 3% to 5%).

In certain embodiments, any of the foregoing compositions of the method further include f) an emulsifying agent (e.g., PEG-40 hydrogenated castor oil) in the amount of 0.01 to 1 percent (e.g., 0.01% to 0.03%, 0.02% to 0.04%, 0.03% to 0.05%, 0.04% to 0.06%, 0.05% to 0.07%, 0.06% to 0.08%, 0.07% to 0.09%, or 0.08% to 0.10%) based on the weight of the composition.

In further embodiments, any of the foregoing compositions of the method include g) a water-miscible alkylene glycol (e.g., propylene glycol) in the amount of 7 to 12 percent (e.g., 7% to 9%, 8% to 10%, 9% to 11%, 10% to 12%) based on the weight of the composition.

In some embodiments, any of the foregoing compositions of the method include h) a cosmetic preservative (benzyl alcohol) in the amount of 0.05 to 0.15 percent (e.g., 0.05% to 0.1%, 0.08% to 0.12%, 0.1 to 0.15%) based on the weight of the composition.

In other embodiments, any of the foregoing compositions of the method include i) an antioxidant or mixture of antioxidants (e.g., Vitamin E, butylated hydroxytoluene, or a mixture thereof) in the amount of 1 to 1.1 percent based on the weight of the composition.

In further embodiments, any of the foregoing compositions of the method include) a pH modifier (e.g., triethanolamine) in an amount sufficient to maintain a pH value of the composition in the range of 4.5 to 6 (e.g., 4.5 to 5, 4.75 to 5.25, 5 to 5.5, 5.25 to 5.75, 5.5 to 6).

In a particular embodiment, the composition of the method includes a topical composition including a) about 10 percent NSAID (e.g., ketoprofen); b) about 1.25 percent of a cross-linked polyacrylic acid interpolymer and about 0.5 percent of a cross-linked polyacrylic acid homopolymer, c) about 19 percent of a mixture of one or more aliphatic alcohols (e.g., a mixture of about 10 percent ethanol and about 9 percent isopropanol) and about 3 percent of an aliphatic ester (e.g., isopropyl myristate); d) about 0.05 percent of a chelating agent (e. g., the disodium salt of ethylenediamineteraacetic acid); e) about 10 percent oxybenzone; f) about 0.5 percent of an emulsifying agent (e.g., PEG-40 hydrogenated castor oil); g) about 10 percent of a water-miscible alkylene glycol (e.g., propylene glycol); h) about 1 percent of a cosmetic preservative (e.g., benzyl alcohol); i) about 1.05 percent of a mixture of antioxidants (e.g., a mixture of about 0.05 percent Vitamin E and 1 percent butylated hydroxytoluene); j) about 1.5 percent of a pH modifier (e.g., triethanolamine); k) and the rest water.

In another embodiment, the composition of the method includes a topical composition including a) about 5 percent NSAID (e.g., ketoprofen); b) about 1.25 percent of a cross-linked polyacrylic acid interpolymer and about 0.5 percent of a cross-linked polyacrylic acid homopolymer; c) about 19 percent of a mixture of one or more aliphatic alcohols (e.g., a mixture of about 10 percent ethanol and about 9 percent isopropanol) and about 3 percent of an aliphatic ester (e.g., isopropyl myristate); d) about 0.05 percent of a chelating agent (e.g., the disodium salt of ethylenediamineteraacetic acid); e) about 5 percent oxybenzone; f) about 0.5 percent of an emulsifying agent (e.g., PEG-40 hydrogenated castor oil); g) about 10 percent of a water-miscible alkylene glycol (e.g., propylene glycol); h) about 1 percent of a cosmetic preservative (e.g., benzyl alcohol); i) about 1.05 percent of a mixture of antioxidants (e.g., a mixture of about 0.05 percent Vitamin E and 1 percent butylated hydroxytoluene); j) about 1.5 percent of a pH modifier (e.g., triethanolamine); k) and the rest water.

In certain embodiments of any of the foregoing compositions of the method, the non-steroidal anti-inflammatory drug is selected from the list consisting of aspirin, diflunisal, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid, celecoxib, rofecoxib, valdecoxib, parecoxib, lumiracoxib, etoricoxib, firocoxib, paracetamol, nimesulide, licofelone, lysine clonixinate, and hyperforin, calcitriol.

In some embodiments, any of the foregoing compositions of the method further include gabapentin in the amount of 8 to 10 percent (e.g., 8% to 9%, 8.5% to 9.5%, 9% to 10%) by weight of the composition.

In some embodiments of any of the foregoing methods, the topical composition does not include benzyl alcohol.

In certain embodiments of any of the foregoing methods, the topical composition is administered in combination with a systemically administered neuroprotective agent (e.g., calcium, magnesium, glutathione, glutamine, carbamaepine, oxycarbazepine, vitamin E, erythropoietin, allopregnanolone, valproate, alpha-lipoic acid, acetyl-L-carnitine or a combination thereof).

In some embodiments of any of the foregoing methods, the peripheral neuropathy is mononeuropathy, mononeuritis multiplex, polyneuropathy, autonomic neuropathy, or neuritis. In other embodiments, the peripheral neuropathy is chemotherapy induced peripheral neuropathy (CIPN) or diabetic neuropathy.

In other embodiments, the symptom associated with sunburn is selected from the group consisting of: blisters, rashes, redness, swelling, tenderness, headaches, fevers, chills, or fatigue.

In a second aspect, the invention features a method for treating peripheral neuropathy or treating pain or a symptom associated with sunburn. This method includes administering to a subject in need thereof an effective amount of a topical composition including: a) a non-steroidal anti-inflammatory drug (e.g., a propionic acid derivative, e.g., ketoprofen, ibuprofen, naproxen, and salts thereof; or an acetic acid derivative, e.g., diclofenac, indomethacin, etodolac, and salts thereof) in an amount of about 1 to about 10 percent by weight (e.g., 1%, 2%, 3%, 4%, 5%, 6%, 7%, 8%, 9%, to 10%); b) lauryl lactate in the amount of about 1 to about 5 percent by weight (e.g., 1%, 2%, 3%, 4%, to 5%); c) lactic acid in an amount of about 0.5 to about 5 percent by weight (e.g., 0.5%, 1%, 1.5%, 2%, 2.5%, 3%, 3.5%, 4%, 4.5%, to 5%); d) glyceryl monolaurate in an amount of about 2 to about 5 percent by weight (e.g., 2%, 3%, 4%, to 5%); and e) a carrier consisting essentially of water and ethanol. Preferably, this composition is a spray.

In some embodiments, the topical composition further comprises propylene glycol (e.g., in an amount of about 5 to about 30 percent by weight (e.g., 5%, 6%, 8%, 10%, 15%, 20%, 25%, to 30%), or a nonionic surfactant having an HLB value of at least 12 (e.g., alkoxylated alcohol) in an amount of about 0 to about 7 percent by weight (e.g., 0.5%, 1%, 1.5%, 2%, 2.5%, 3%, 3.5%, 4%, 4.5%, 5%, 5.5%, 6%, 6.5%, to 7%).

In another embodiment, the water:ethanol weight ratio of said carrier is in the range of about 0.3:1 to about 2.6:1 (0.3:1, 0.5:1, 0.8:1, 1:1, 1.3:1, 1.5:1, 1.8:1, 2.0:1, 2.2:1, 2.3:1, to 2.6:1).

In yet another embodiment, the topical composition includes: a) about 5 percent by weight ketoprofen; b) about 3 percent by weight lauryl lactate; c) about 1.5 percent by weight lactic acid; d) about 3 percent by weight glyceryl monolaurate; e) about 3 percent by weight polyethylene glycol having a HLB value of about 15.7; f) about 10 percent by weight propylene glycol; and g) a water:ethanol weight ratio of about 1.7.

As used herein, the term “administration” or “administering” refers to a method of giving a dosage of a pharmaceutical composition to a subject. The preferred method of administration may depend on a variety of factors, e.g., the components of the pharmaceutical composition and the nature and severity of the disease, disorder, or condition. The phrase “administered in combination” means administering to the patient a pair of active agents, the agents may be administered within 28, 21, 14, 10, 7, 5, 4, 3, 2, or 1 days; within 24, 12, 6, 3, 2, or 1 hours; or substantially simultaneously.

As used herein, the term “anesthetic” refers to a compound or composition that result in anesthesia (i.e., reversible loss of sensation). Examples of include amethocaine, cocaine, prilocaine, bupivacaine, levobupivacaine, ropivacaine, mepivacaine, dibucaine, benzocaine, physostigmine, neostigmine, lidocaine, novocaine and procaine.

The phrase “dermatologically acceptable” means that the compositions or components thereof are suitable for use in contact with dermal tissue without undue toxicity, incompatibility, instability, allergic response, and the like.

The expression “effective amount” as used herein, refers to a sufficient amount of a composition of the invention to exhibit the desired therapeutic effect. The exact amount required will vary from subject to subject, depending on the species, age, and general condition of the subject, the particular therapeutic agent and the like. The compositions of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compositions of the present invention will be decided by the attending physician within the scope of sound medical judgment. The specific therapeutically effective dose level for any particular patient or organism will depend upon a variety of factors including the condition being treated and the severity of the condition; the activity of the specific compounds employed; the specific composition employed; the age, body weight, general health, sex and diet of the patient; the time of administration, route of administration, and rate of excretion of the specific compounds employed; the duration of the treatment; drugs used in combination or coincidental with the specific composition employed; and like factors well known in the medical arts.

By “palliation” of a disease, a disorder, or a condition is meant that the extent and/or undesirable clinical manifestations of the disease, disorder, or condition are lessened and/or the time course of the progression is slowed or lengthened, as compared to the extent or time course in the absence of treatment.

By “subject” is meant any animal (e.g., a primate, e.g., a human). Any animal can be treated using the methods, compositions, and kits of the invention.

As used herein, the term “treatment” refers to administering a composition of the invention or a combination therapy of the invention for therapeutic purposes. To “prevent or inhibit disease” refers to prophylactic treatment of a subject who is not yet ill, but who is susceptible to, or otherwise at risk of, a particular disease or condition. To “treat a condition” or use for “therapeutic treatment” refers to administering treatment to a subject already suffering from a condition to improve or stabilize the subject's condition. Beneficial or desired results can include, but are not limited to, alleviation or amelioration of a disease, a disorder, a condition, or one or more symptoms associated with a disease, a disorder, or a condition; diminishment of extent of disease, disorder, or condition; stabilization (i.e., not worsening) of a disease, disorder, or condition; delay or slowing the progress of a disease, disorder, or condition; and amelioration or palliation of a disease, disorder, or condition. “Treatment” can also mean prolonging survival as compared to expected survival if not receiving treatment. By “prevention” is meant that a prophylactic treatment is given to a subject who has or will have a disease, a disorder, a condition, or one or more symptoms associated with a disease, a disorder, or a condition.

### BRIEF DESCRIPTION OF THE DRAWINGS

FIG. 1A is a chart illustrating the effect of ELS-M11 against taxol-mediated nerve pain in mice, as measured by automated measurement of the avoidance in von Frey hair test.

FIG. 1B is a chart illustrating the effect of ELS-M11 against taxol-mediated nerve pain in mice, as measured by cold allodynia behavior in an acetone evaporative cooling test.

### DETAILED DESCRIPTION OF THE INVENTION

The present disclosure relates to compositions and methods for the treatment of peripheral neuropathies. In particular, the present disclosure provides dermatologically acceptable compositions for topical administration that can alleviate the symptoms of peripheral neuropathies (e.g., chemotherapy induced peripheral neuropathy) or treating pain or a symptom associated with sunburn. The invention also provides combinations of these topical compositions and systemically administered neuroprotective agents to minimize the impact of chemotherapy induced peripheral neuropathy to improve the tolerability of chemotherapeutic agents. The methods and compositions of the invention can be used to treat peripheral neuropathy either by preventing the occurrence of pain or by treating one or more symptoms associated with peripheral neuropathy. The invention can be used to treat any type of peripheral neuropathy, including chemotherapy-induced peripheral neuropathy, diabetic neuropathy, or idiopathic neuropathy. Symptoms and diagnosis of neuropathy can be readily determined by any parameters well known in the art, for example, as described in Leeds Assessment of Neuropathic Symptoms and Signs (LANSS), Douleur neuropathique 4 questions (DN4), the Neuropathic Pain Questionnaire (NPQ).

### Peripheral Neuropathy

Peripheral neuropathy resulting from chemotherapy or other causes may manifest itself in a variety of ways. In addition, peripheral neuropathy from a single etiology may manifest differently in different patients. The most common is with sensory disturbances, including both the negative symptom of numbness and the positive symptoms of pain and parasthesias. The sensory symptoms most often start in the distal extremities in a ‘glove-and-stocking’ distribution. Motor symptoms may occur, usually manifesting as distal weakness such as a foot drop. In addition, autonomic symptoms may occur which can include orthostatic, cardiovascular, erectile or gastrointestinal disturbances. Autonomic neuropathy may be the hardest to diagnose accurately, and often these symptoms may occur in isolation and not in the presence of a generalized autonomic failure. For example, isolated orthostatic hypotension may be the only autonomic manifestation of vincristine neuropathy. However, autonomic dysfunction usually occurs in the setting of wide-spread peripheral neuropathy, which can range from mild to prominent.

Peripheral neuropathy is classified according to the number of nerves affected, the type of nerve affected, or the process affecting the nerve.

### Mononeuropathy

Mononeuropathy is the classification for peripheral neuropathy that affects a single nerve. The most common causes for mononeuropathy are a localized trauma or infection, such as compression of the nerve. Direct injury to a nerve, interruption of the blood supply of a nerve (i.e., ischemia), or inflammation are other causes of mononeuropathy. Examples of conditions classified as mononeuropathies are carpal tunnel syndrome and other nerve compression syndromes, axillary nerve palsy, and ulnar neuropathy.

### Mononeuritis Multiplex

Mononeuritis multiplex (or polyneuritis multiplex) are peripheral neuropathies in which individual noncontiguous nerve trunks are simultaneously or sequentially involved. Mononeutits multiplex may involve deep, aching pain in the lower back, hip, or leg. Conditions that cause or are associated with mononeuritis multiplex include, diabetes mellitus, vasculitides, immune-mediated diseases such as rheumatoid arthritis, lupus erythematosus, and sarcoidosis, infections such as, leprosy, lyme disease, and human immunodeficiency virus, amyloidosis, cryoglobulinemia, exposure to certain chemical agents such as trichloroethylene and dapsone.

### Polyneuropathy

Polyneuropathy is a pattern of nerve damage wherein many nerve cells in various parts of the body are affected. Polyneuropathy generally presents in one of three patterns, a) distal axonopathy, wherein the cell bodies of the neurons remain intact, but the axons are affected, a common pattern in diabetic neuropathy; b) demyelinating, wherein the myelin sheath around axons is damaged, affecting the ability of axons to control electrical impulses; and c) motor neuron disease or sensory neuronopathy wherein the cell bodies of the neurons are directly affected. Diabetes is the most common cause of polyneuropathy. Other causes include lyme disease, vitamin deficiencies, blood disorders, and exposure to toxins.

### Autonomic Neuropathy

Autonomic neuropathy is a particular form of polyneuropathy wherein the non-voluntary, non-sensory nervous system is affected. Autonomic neuropathy generally presents in combination with another form of neuropathy. Autonomic neuropathy generally affects internal organs such as the bladder, cardiovascular system, the gastrointestinal tract, and genital organs.

### Neuritis

Neuritis results from inflammation of a nerve or the peripheral nervous system in general. Symptoms of neuritis include pain, paresthesia, paresis, hypoesthesia, anesthesia, paralysis, wasting, and loss of relexes. Neuritis can have many causes, some exemplary causes are physical injury, infection (e.g., herpes simplex, shingles, leprosy, lyme disease), exposure to chemicals or radiation, diphtheria, diabetes, vitamin deficiencies, autoimmune disease (e.g., multiple sclerosis, Guillian-Barre syndrome), and cancer. Specific types of neuritis include polyneuritis, brachial neuritis, optic neuritis, vestibular neuritis, cranial neuritis, and arsenic neuritis.

### Chemotherapy Induced Peripheral Neuropathy (CIPN)

Chemotherapy induced peripheral neuropathy is a condition that results from damage to peripheral nerves by chemotherapy drugs used in the treatment of various diseases (e.g., cancer). The most common symptoms of CIPN are pain, burning, tingling, loss of feeling, loss of dexterity, balance problems, increased sensitivity to pressure and/or temperature, decreased muscle mass and/or muscle weakness, trouble swallowing, constipation, retention of urine, blood pressure changes, and loss or lack of reflexes. CIPN generally affects both sides of the body equally (e.g., the stocking-glove distribution) and can begin anytime after treatment starts. The degree of severity of CIPN depends not only on the drug, duration, and dose used but also on the total cumulative dose applied.

Depending on the substance used, a pure sensory syndrome (e.g., with cisplatin, oxaliplatin, carboplatin) or a mixed sensory-motor neurotoxicity with or without involvement of the autonomic nervous system (e.g., vincristine, taxol) can ensue. In addition, previous administration or co-treatment with two or more neurotoxic agents further increases the incidence and severity of neurotoxic effects. For example, cisplatin alone induced neurotoxic effects in 49% of patients (Bacon M. and et al. 2003, Int. J. Gynecol. Cancer 13:428-34), whereas when co-administered with paclitaxel, sensory neurotoxicity was observed in 91% of patients. These effects may lead to disability and worsening of life quality in the absence of tumor progression and it represents a serious dose-limiting side effect. In addition, the development of neurotoxic syndrome can interfere with optimal drug dosing, delay sequencing of therapy, or necessitate the discontinuation of treatment.

Vincristine-driven neuropathy is mainly characterized by motor and sensory insufficiency (mixed type of neuropathy). While the underlying mechanism is not fully understood as yet, it has been described to involve an alteration of anterograde axonal transport, ultimately leading to axonal degeneration.

Cis-dichlorodiamine platinum (cisplatin) is the drug of choice for treatment of germ cell cancers. It is also used adjunctively for other solid tumors but the total dose that can be administered is limited by serious adverse effects including renal toxicity and peripheral neurotoxicity. The incidence of nephropathy, which was dose limiting, has been significantly reduced by chloride diuresis. Problems of peripheral neuropathy appear soon after the drug is introduced. The neuropathy is dose limiting and closely related to total cumulative drug dose. Significant peripheral neurotoxicity is apparent in the majority of adult patients who receive >400-500 mg/m² of cisplatin. The neuropathy is predominantly sensory with initial complaints of paresthesiae (abnormal sensation as burning, prickling, formication) in the distal extremities, which may progress to severe sensory ataxia. Neuropathological studies have shown loss of large myelinated fibers and evidence of axonal degeneration. The neuropathy may continue to progress for several months after cessation of cisplatin and symptoms may develop 3-8 wk after the last dose of chemotherapy (Thompson et al., (1984) Cancer. 54(7): 1269-75). Studies of tissue platinum assays, monitoring the tissues in which platinum accumulates, revealed the highest platinum concentration in tumor tissue, but similarly high concentrations were found in peripheral nervous tissue. This compared with much lower concentrations in brain. Electrophysiological studies in cancer patients treated with cisplatin confirm that large diameter sensory axons are involved.

Taxol is an effective chemotherapeutic agent extensively used in the treatment of solid tumors such as malignant melanoma and ovarian carcinoma. Nevertheless, peripheral neuropathy caused by taxol is increasingly becoming a dose-limiting problem in cancer treatment (Rowinsky E. K., Chaudhry V., Comblath D. R, Donehower R. C. Neurotoxicity of taxol (1993). Monogr. Natl. Cancer Inst. 107-115.). Taxol is a plant alkaloid that suppresses microtubule dynamics through binding to tubulin subunits, causing mitotic arrest in dividing cells (Derry W. B, Wilson L., Jordan M. A. Substoichiometric binding of taxol suppresses microtubule dynamics (1995) Biochemistry 34: 2203-2211.), and axonal degeneration in peripheral nerves due to interference with axonal transport (Rowinsky et al., 1993). The resulting neuropathy predominantly affects small sensory fibers, but at higher doses motor and large sensory fiber dysfunction occur (Freilich R. I., Balmaceda C., Seidman A. D., Rubin M., DeAngelis L. M. Motor neuropathy due to docetaxel and paclitaxel (1996). Nutr. Rev. 47: 115-118).

The most sensitive and reliable method of detecting CIPN is by history and physical examination, with specific attention to questions about the symptoms of neuropathy, such as numbness, tingling, pain, or distal weakness, and the physical signs of reflexes (especially the ankle jerks), motor and sensory examinations. Other quantitative examinations, such as nerve conduction studies (NCS) and electromyography (EMG) or quantitative sensory threshold testing, may play a role in further evaluation and differentiating CIPN from other potential etiologies, but are rarely necessary for initial detection (Ocean & Vahdat, 2004). These techniques may also be important in clinical trials of CIPN (DeAngelis et al, 1991; Forsyth et al, 1997; Postma & Heimans, 2000). Of these ancillary techniques, NCS and EMG are probably the most useful. NCS measure the latency, velocity and amplitude of impulses travelling along a nerve. Different types of injuries cause different patterns of change on NCS. Axonal damage, (e.g., caused by a chemotherapeutic such as vincristine), manifests as decreased amplitude on NCS. Decreased conduction velocity or increased distal latency would suggest a demyelinating polyneuropathy, as seen in the neuropathy associated with multiple myeloma cancer, such a chronic inflammatory demyelinating polyneuropathy, which requires specific therapy or a polyradiculopathy from metastatic disease, which can be differentiated from peripheral neuropathy by NCS/EMG. Lastly, NCS/EMG may help to determine the degree of nerve damage from CIPN, which may be used as a baseline and can then be followed if new symptoms develop.

Pain and Symptoms Associated with Sunburn

The compositions of the invention are also useful in treating pain and symptoms associated with sunburn. Sunburn results from too much sun or sun-equivalent exposure. Mild and uncomplicated cases of sunburn usually result in minor skin redness and pain. Initially, the skin turns red about 2 to 6 hours after exposure and feels irritated, the peak effects are noted at 12 to 24 hours. More severe cases of sunburn, also called sun poisoning are complicated by severe skin burning and blistering, massive fluid loss (dehydration), electrolyte imbalance, and possible infection. With too much exposure, severe untreated sunburn can cause shock or poor circulation to vital organs. The compositions of the invention are useful for treating the symptoms of sunburn. Symptoms of sunburn include, but are not limited to: blisters, rashes, skin loss, pinkness or redness in skin, skin that feels warm or hot to the touch, tenderness headache, fever, chills and fatigue.

In some embodiments, administration of the compositions described herein to a subject with sunburn will speed up the recovery process and healing from the sunburn (e.g., by 2 days, 3 days. 4 days, 5 days, 6 days. 7 days, 8 days, 9 days) as compared to subjects treated by another method known in the art for treatment of sunburns or untreated subjects. The healing of sunburns can be assessed by any standard method known in the art, for example, visual minimal erythema dose (MED) readings, skin reaction measurements, constitutive colorimetric readings, or assessment of melanin pigmentation after UV irradiation by laser scanning confocal microscopy.

### Therapeutic Agents

### NSAIDs

A class of compounds useful in the invention are the non-Steroidal Anti-Inflammatory Drugs (NSAIDs), which provide analgesic and antipyretic (fever-reducing) effects, and, in higher doses, anti-inflammatory effects. The term “nonsteroidal” distinguishes these drugs from steroids, which, among a broad range of other effects, have a similar eicosanoid-depressing, anti-inflammatory action. As analgesics, NSAIDs are unusual in that they are non-narcotic. NSAIDs are usually indicated for the treatment of acute or chronic conditions where pain and inflammation are present. The widespread use of NSAIDs has meant that the adverse effects of these drugs have become increasingly prevalent. The two main adverse drug reactions associated with NSAIDs relate to gastrointestinal effects and renal effects of the agents. NSAIDs can be classified based on their chemical structure or mechanism of action. Common NSAID classification groups include: Salicylates (e.g., aspirin (acetylsalicylic acid), diflunisal, and salsalate), Propionic acid derivatives (e.g., ibuprofen, dexibuprofen, naproxen, fenoprofen, ketoprofen, dexketoprofen, flurbiprofen, oxaprozin, and loxoprofen). Acetic acid derivatives (e.g., indomethacin, tolmetin, sulindac, etodolac, ketorolac, diclofenac, and nabumetone), Enolic acid derivatives (e.g., piroxicam, meloxicam, tenoxicam, droxicam, lornoxicam, and isoxicam), Fenamic acid derivatives (e.g., mefenamic acid, meclofenamic acid, flufenamic acid, and tolfenamic acid), Selective COX-2 inhibitors (e.g., celecoxib, rofecoxib, valdecoxib, parecoxib, lumiracoxib, etoricoxib, firocoxib, and paracetamol), and Sulphonanilides (e.g., nimesulide), along with other compounds that do not fall into one of these categories (e.g., licofelone, lysine clonixinate, and hyperforin, and calcitriol). NSAIDs within a group tend to have similar characteristics and tolerability. There is little difference in clinical efficacy among the NSAIDs when used at equivalent doses. Rather, differences among compounds usually relate to dosing regimens, route of administration, and tolerability profile.

One preferred compound useful in the invention is ketoprofen is a non-steroidal propionic acid derivative. It has potent anti-inflammatory and analgesic activity. Conventionally, ketoprofen and other related drugs have been administered orally; however, this can be accompanied by systemic side effects and/or gastrointestinal irritation. In order to reduce these side effects, these drugs have been formulated as transdermal preparations. The skin permeability of these NSAIDs is known to be higher than other NSAIDs.

### Anesthetics

Another class of compounds useful in the invention are anesthetics, which are compounds or compositions that result in anesthesia (i.e., reversible loss of sensation). They differ from analgesics (e.g., NSAIDs) in that analgesics relieve pain without eliminating sensation. Anesthetics are classified into two groups: general anesthetics which result in a loss of consciousness and local anesthetics which result in a loss of sensation for a limited region of the body. Examples of local anesthetics include amethocaine, cocaine, prilocaine, bupivacaine, levobupivacaine, ropivacaine, mepivacaine, dibucaine, benzocaine, physostigmine, neostigmine, lidocaine, novocaine and procaine.

### Neuroprotective Agents

Also useful in the methods of the invention are neuroprotective agents, which are compounds and compositions administered to prevent or slow the damage or loss of neurons. Several neuroprotective agents have been tested for the prevention of CIPN with varying results (Pachman et al. Clinical Pharmacology & Therapeutics, 90(3):377-386 (2011)); these include, without limitation, calcium, magnesium, glutathione, glutamine, carbamaepine, oxycarbazepine, vitamin E, erythropoietin, allopregnanolone, valproate, alpha-lipoic acid, acetyl-L-carnitine and combinations, such as calcium and magnesium infusions.

### Dosage, Formulation, and Administration

The compositions of the invention may conveniently be administered in unit dosage form and may be prepared by any of the methods well-known in the pharmaceutical art, for example, as described in “Remington: The Science and Practice of Pharmacy” (20th ed., ed. A. R. Gennaro, 2000, Lippincott Williams & Wilkins). The concentration of at least one therapeutic agent in the formulation will vary depending upon a number of factors, including the dosage of the drug to be administered, and the route of administration.

The composition can be prepared in any useful method. For example, at least one therapeutic agent is dissolved in ethanol and added to a mixture of polyethylene glycols (PEGs). In another example, the composition further includes a skin penetrating enhancer of a dimethyl alanine amide of medium chain fatty acids with carbon units varying between C₁₂ and C₁₆. More specifically, therapeutic agents alone or combinations thereof may be prepared in an ointment, cream, lotion, foam or spray form. In these forms, unit dispensing would be preferred, where the unit dosage of the therapeutic agent and vehicle would be in the range of 100 mg to 1000 g and most preferred between 100 mg and 500 mg. The therapeutic agent in this composition by weight would be in the range of 1% to 30% (w/w). The most preferred range would be between 5% and 10% (w/w). In another embodiment, the composition comprises between 1%-3%, 2.5%-6%, 8%-12%, 10%-20%, or 20-30% (w/w) of at least one therapeutic agent. In one implementation, the therapeutic agent is present in the composition in an amount of at least 0.5%, at least 2%, at least 2.5%, at least 3%, at least 5%, or at least 10%, (w/w), and may be, for example, ketoprofen. In some embodiments, the concentration of a therapeutic agent is less than 10% (w/w) (e.g., 1% to 9%, 1% to 8%, 1% to 7%, 1% to 6%, 1% to 5%, 2% to 9%, 2% to 8%, 2% to 7%, 2% to 6%, 2% to 5%, 3% to 9%, 3% to 8%, 3% to 7%, 3% to 6%, or 3% to 5%).

### Dosages

The dosage of a compound or a combination of compounds depends on several factors, including: the administration method, the type of condition to be treated, the severity of the condition, whether administration first occurs at an early or late stage of the condition, and the age, weight, and health of the patient to be treated. For combinations that include a synergistic pair of agents identified herein, the recommended dosage for the agents can be less than or equal to the recommended dose as given in the Physician's Desk Reference, 60^{th} Edition (2006). An agent is usually given by the same route of administration that is known to be effective for delivering it as a monotherapy. When used in combination therapy according to the methods of this invention, an agent is dosed in amounts and frequencies equivalent to or less than those that result in its effective monotherapeutic use.

Optimization of the appropriate dosages can readily be made by the skilled practitioner in light of the pharmacokinetics of the therapeutic agent or combination of therapeutic agents used in the composition. Factors to be considered in setting dosages include the therapeutic agent's specific activity; the severity of the condition or symptoms of the subject; the age, condition, body weight, sex, and diet of the subject; the use (or not) of concomitant therapies; and other clinical factors.

### Delivery of Compound(s)

It is not intended that administration of compounds be limited to a single formulation or delivery method for all compounds of a combination. The combination can be administered using separate formulations and/or delivery methods for each compound of the combination using, for example, any of the above-described formulations and methods. In one example, a first agent is delivered topically, and a second agent is delivered orally.

### Formulation

The compositions can be provided in any useful form. For example, the compositions of the invention may be formulated as solutions, emulsions (including micro emulsions), suspensions, sprays, creams, foams, lotions, gels, powders, or other typical solid, semi-solid, or liquid compositions used for application to the skin or other tissues where the compositions may be used. The preferred compositions may also be applied as a patch. Such compositions may contain other ingredients typically used in such products, such as colorants, fragrances, thickeners (e.g., xanthan gum, a fatty acid, a fatty acid salt or ester, a fatty alcohol, a modified cellulose, a modified mineral material, Krisgel 100™, or a synthetic polymer), antimicrobials, solvents, surfactants, detergents, gelling agents, antioxidants, fillers, dyestuffs, viscosity-controlling agents, preservatives, humectants, emollients (e.g., natural or synthetic oils, hydrocarbon oils, waxes, or silicones), hydration agents, chelating agents, demulcents, solubilizing excipients, adjuvants, dispersants, skin penetrating enhancers, plasticizing agents, preservatives, stabilizers, demulsifiers, wetting agents, sunscreens, emulsifiers, moisturizers, astringents, deodorants, and optionally including anti-itch actives, botanical extracts, conditioning agents, darkening or lightening agents, glitter, humectants, mica, minerals, polyphenols, silicones or derivatives thereof, sunblocks, vitamins, and phytomedicinals.

The compositions can also include other like ingredients to provide additional benefits and improve the feel and/or appearance of the topical formulation. Specific classes of additives commonly use in these formulations include: isopropyl myristate, sorbic acid NF powder, polyethylene glycol, phosphatidylcholine (including mixtures of phosphatidylcholine, such as phospholipon G), Krisgel 100™, distilled water, sodium hydroxide, decyl methyl sulfoxide (as a skin penetrating enhancer), menthol crystals, butylated hydroxytoluene, ethyl diglycol reagent, and 95% percent (190 proof) ethanol.

The preferred compositions are sprays. Sprays are advantageous for patients having peripheral neuropathy relative to other topical formulations (e.g., gels and creams), as application of gels and creams to the skin of such patients can be painful. The compositions of the invention can have low dynamic viscosity (e.g., less than 1000 cP at 25° C. or less than 900 cP at 25° C.). The dynamic viscosity of the compositions of the invention can be at least 1 cP at 25° C. (e.g., at least 2 cP, at least 10 cP, at least 20 cP, at least 50 cP, or at least 100 cP). Non-limiting, exemplary ranges of the dynamic viscosity of the compositions of the invention are: 1 cP to 1000 cP, 1 cP to 900 cP, 2 cP to 1000 cP, 2 cP to 900 cP, 10 cP to 1000 cP, 10 cP to 900 cP, 20 cP to 1000 cP, 20 cP to 900 cP, 50 cP to 1000 cP, 50 cP to 900 cP, 100 cP to 1000 cP, or 100 cP to 900 cP). In preferred embodiments, the compositions of the invention have a high water content (e.g., at least 15% (w/w), at least 20% (w/w), or at least 25% (w/w)). Non-limiting, exemplary ranges for the water content of the compositions of the invention are: from 15% (w/w) to 60% (w/w), from 15% (w/w) to 50% (w/w), from 20% (w/w) to 60% (w/w), from 20% (w/w) to 50% (w/w), from 25% (w/w) to 60% (w/w), from 25% (w/w) to 50% (w/w), from 30% (w/w) to 60% (w/w), from 30% (w/w) to 50% (w/w), from 35% (w/w) to 60% (w/w), from 35% (w/w) to 50% (w/w), from 40% (w/w) to 60% (w/w), and from 40% (w/w) to 50% (w/w). High water content of the preferred compositions of the invention may be advantageous for the delivery of a therapeutic agent.

Preferably the compositions of the invention do not leave an appreciable residue (e.g., leave no film).

### Pharmaceutically Acceptable Salts

The therapeutic agents may be optionally administered as a pharmaceutically acceptable salt, such as a non-toxic acid addition salts or metal complexes that are commonly used in the pharmaceutical industry. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts, include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Carriers

The compositions can be formulated using any dermatologically acceptable carrier. Exemplary carriers include a solid carrier, such as alumina, clay, microcrystalline cellulose, silica, or talc; and/or a liquid carrier, such as water, an alcohol, a glycol, or a water-alcohol/glycol blend. The therapeutic agents may also be administered in liposomal formulations that allow therapeutic agents to enter the skin. Such liposomal formulations are described in U.S. Pat. Nos. 5,169,637; 5,000,958; 5,049,388; 4,975,282; 5,194,266; 5,023,087; 5,688,525; 5,874,104; 5,409,704; 5,552,155; 5,356,633; 5,032,582; 4,994,213; and PCT Publication No. WO 96/40061. Examples of other appropriate vehicles are described in U.S. Pat. No. 4,877,805 and EP Publication No. 0586106A1. Suitable vehicles of the invention may also include mineral oil, petrolatum, polydecene, stearic acid, isopropyl myristate, polyoxyl 40 stearate, stearyl alcohol, or vegetable oil.

Skin Penetration Enhancers

The composition can further include a skin penetrating enhancer, such as those described in “Percutaneous Penetration Enhancers”, (eds. Smith E W and Maibach H I. CRC Press 1995). Exemplary skin penetrating enhancers include alkyl (N,N-disubstituted amino alkanoate) esters, such as dodecyl 2-(N,N dimethylamino) propionate (DDAIP), which is described in patent U.S. Pat. Nos. 6,083,996 and 6,118,020, which are both incorporated herein by reference; a water-dispersible acid polymer, such as a polyacrylic acid polymer, a carbomer (e.g., Carbopol™ or Carbopol 940P™, available from B. F. Goodrich Company (Akron, Ohio)), copolymers of polyacrylic acid (e.g., Pemulen™ from B. F. Goodrich Company or Polycarbophil™ from A. H. Robbins. Richmond, Va.: a polysaccharide gum, such as agar gum, alginate, carrageenan gum, ghatti gum, karaya gum, kadaya gum, rhamsan gum, xanthan gum, and galactomannan gum (e.g., guar gum, carob gum, and locust bean gum), as well as other gums known in the art (see for instance, Industrial Gums: Polysaccharides & Their Derivatives, Whistler R. L., BeMiller J. N. (eds.), 3rd Ed. Academic Press (1992) and Davidson, R. L., Handbook of Water-Soluble Gums & Resins, McGraw-Hill, Inc., N.Y. (1980)); or combinations thereof.

A “cross-linked polyacrylic acid homopolymer” suitable for present purposes is a high molecular weight polymer of acrylic acid cross-linked with polyalkenyl ethers of sugars or polyalcohols such as allyl sucrose, allyl pentacrythiritol, etc., such as Carbopol® 980 NF and the like. Carbopol® 980 NF is commercially available from Lubrizol Advanced Materials, Inc., Cleveland, Ohio. A “cross-linked polyacrylic interpolymer” suitable for present purposes is a high molecular weight copolymer of acrylic acid and C₁-C₂₄ alkylmethyacrylates cross-linked with polyalkenyl ethers of sugars or polyalcohols which contain a heterologous polymer, e.g., a block copolymer of polyethylene glycol and a long chain, e.g., C₁-C₂₄ alkyl acid ester, such as Carbopol® Ultrez 10 NF, and the like. Carbopol® Ultrez NF is commercially available from Lubrizol Advanced Materials, Inc., Cleveland. Ohio.

Other suitable polymeric skin penetrating enhancers are cellulose derivatives, such as ethyl cellulose, methyl cellulose, hydroxypropyl cellulose. Additionally, known transdermal penetrating enhancers can also be added, if desired. Illustrative are dimethyl sulfoxide (DMSO) and dimethyl acetamide (DMA), 2-pyrrolidone, N,N-diethyl-m-toluamide (DEET), 1-dodecylazacycloheptane-2-one (Azone™, a registered trademark of Nelson Research). N,N-dimethylformamide, N-methyl-2-pyrrolidone, calcium thioglycolate and other enhancers such as dioxolanes, cyclic ketones, and their derivatives.

Also illustrative are a group of biodegradable absorption enhancers which are alkyl N,N-2-(disubstituted amino) alkanoates as described in U.S. Pat. No. 4,980,378 and U.S. Pat. No. 5,082,866, which are both incorporated herein by reference, including: tetradecyl (N,N-dimethylamino) acetate, dodecyl (N,N-dimethylamino) acetate, decyl (N,N-dimethylamino) acetate, octyl (N,N-dimethylamino) acetate, and dodecyl (N,N-diethylamino) acetate.

Examples of skin penetrating enhancers useful in the present invention include isopropyl myristate; isopropyl palmitate; dimethyl sulfoxide; decyl methyl sulfoxide; dimethylalanine amide of a medium chain fatty acid; dodecyl 2-(N,N-dimethylamino) propionate or salts thereof, such as its inorganic (e.g., hydrochloric, hydrobromic, sulfuric, phosphoric, and nitric acid addition salts) and organic salts (e.g., acetic, benzoic, salicylic, glycolic, succinic, nicotinic, tartaric, maleic, malic, pamoic, methanesulfonic, cyclohexanesulfamic, picric, and lactic acid addition salts), as described in U.S. Pat. No. 6,118,020; and alkyl 2-(N,N-disubstituted amino)-alkanoates, as described in U.S. Pat. No. 4,980,378 and U.S. Pat. No. 5,082,866.

The skin penetrating enhancer in this composition by weight would be in the range of 0.5% to 10% (w/w). The most preferred range would be between 1.0% and 5% (w/w). In another embodiment, the skin penetrating enhancer comprises between 0.5%-1%, 1%-2%, 2%-3%, 3%-4%, or 4%-5%, (w/w) of the composition.

Emulsifying Agents

Emulsifying agents are used to stabilize emulsions and include both a hydrophobic and hydrophilic component in their chemical structure. They can also aid in the dissolution of ingredients in a solvent in which they would not normally dissolve. Emulsifying agents generally concentrate at and are adsorbed onto the oil:water interface to provide a protective barrier around the dispersed droplets. Exemplary emulsifying agents include tragacanth, triethanolamine oleate, potassium oleate, sodium oleate, sodium lauryl sulfate, sodium dioctyl sulfosuccinate, glyceryl monostearate, and polymers, such as Spans® (e.g., Span® 20, 40, 60, 65, 80, 85), Tweens® (e.g., Tween® 20, 21, 40, 60, 61, 65, 80, 81, 85), PEG 400 Monoleate, PEG 400 monostearate, PEG 400 monolauarate, and PEG-40 hydrogenated castor oil (a polyethylene glycol derivative of castor oil). Emulsifying agents can be classified according to chemical structure or mechanism of action. All emulsifying agents must be chemically stable in the system, inert, and chemically non-reactive with other components of the composition, and nontoxic and nonirritant. Examples of synthetic emulsifying agents include benzalkonium chloride, benzethonium chloride, alkali soaps (e.g., sodium or potassium oleate), amine soaps (e.g., triethanolamine stearate), detergents (e.g., sodium lauryl sulfate, sodium dioctyl sulfosuccinate, sodium docusate), sorbitan esters (Spans®), polyoxyethylene derivatives of sorbitan esters (Tweens®), or glyceryl esters. Examples of hydrocolloid emulsifying agents include vegetable derivatives (e.g., acacia, tragacanth, agar, pectin, carrageenan, lecithin), animal derivatives (e.g., gelatin, lanolin, cholesterol), semi-synthetic agents (e.g., methylcellulose, carboxymethylcellulose), and synthetic agents (e.g., Carbopols®). Examples of solid particle emulsifying agents include bentonite, veegum, hectorite, magnesium hydroxide, aluminum hydroxide and magnesium trisilicate.

Water-Miscible Alkylene Glycols

Suitable water-miscible alkylene glycols are polyhydric alcohols such as glycerol, dipropylene glycol, polyethylene glycol, propylene carbonate, propylene glycol, butylene glycol, pentylene glycol, hexylene glycol, and the like. Propylene glycol is the preferred water-miscrible alkylene glycol.

Propylene glycol is a colorless, nearly odorless, clear, viscous liquid. Propylene glycol acts as a solvent and antimicrobial in the present formulation. The freezing point of water is depressed when mixed with propylene glycol due to increased opportunity for hydrogen bonding. The total propylene glycol concentration should not exceed 12% w/w to avoid a negative effect on permeation and physical stability.

Lipophilic Solvent

Lipophilic solvents of the present invention may contain a mixture of one or more aliphatic alcohols (e.g., C₂ to C₈ monohydric alkanol alcohols) and one or more C₈ to C₃₀ aliphatic esters. Alternatively, a lipophilic solvent may be one or more C₈ to C₃₀ aliphatic esters. Suitable monohydric alkanol alcohols are C₂ and C₃ alkanols such as ethanol, propanol, isopropanol, and the like. Isopropyl alcohol and ethyl alcohol are colorless, flammable, chemical compounds. They are miscible in water, alcohol, ether and chloroform. Isopropyl alcohol and ethyl alcohol dissolve a wide range of non-polar compounds. They also evaporate quickly and are relatively non-toxic, compared to alternative solvents. In the present composition, isopropyl alcohol and ethyl alcohol act as solvent and permeation enhancers. Suitable aliphatic esters are lauryl lactate, propylene glycol laurate, isopropyl myristate, isopropyl palmitate, propylene glycol caprylate, lanolin, and the like. Lauryl lactate, propylene glycol laurate, and isopropyl myristate are the preferred aliphatic esters. Lauryl lactate and propylene glycol laurate are the particularly preferred aliphatic esters. In compositions containing isopropyl myristate, isopropyl myristate can act as a solvent, stabilizer, as well as an emollient.

In some embodiments, the composition of the invention contains 10% (w/w) to 60% (w/w) of the one or more aliphatic alcohols (e.g., 10% (w/w) to 50% (w/w), 10% (w/w) to 40% (w/w), 20% (w/w) to 60% (w/w), 20% (w/w) to 50% (w/w), or 20% (w/w) to 40% (w/w)).

In certain embodiments, the composition of the invention contains 1% (w/w) to 10% (w/w) of the one or more aliphatic esters (e.g., 1% (w/w) to 6% (w/w), 2% (w/w) to 10% (w/w), 2% (w/w) to 6% (w/w), 3% (w/w) to 10% (w/w), or 3% (w/w) to 6% (w/w)).

The alcohols present in the compositions contribute to skin permeation; however, the total monohydric alcohol concentration is preferably 50% (w/w) or less (e.g., 45% (w/w) or less, 40% (w/w) or less, or 35% (w/w) or less) to maintain optimum skin permeation.

Cosmetic Preservative

The composition can further include a cosmetic preservative, such as those described in “Preservatives for Cosmetics, 3^{rd} Edition”, (Steinberg, D. C. Allured Pub. Corp. 2012). Suitable cosmetic preservatives are the parabens such as methylparaben, propylparaben, butylparaben, phenol derivatives such as phenoxyethanol, benzyl alcohol, and the like. Benzyl alcohol is the preferred preservative. Benzyl alcohol is partially soluble in water and completely miscible in alcohols and diethyl ether. Benzyl alcohol acts as a bacteriostatic preservative in the present compositions.

Oxybenzone

Oxybenzone ((2-Hydroxy-4-methoxyphenyl)-phenylmethanone), molecular formula C₁₄H₁₂O₃, absorbs UVB and UVA (ultraviolet) radiation. It forms coloriess crystals that are readily soluble in most organic solvents and contributes to the photostability of the composition.

Antioxidants

Antioxidants are included in pharmaceutical formulations to enhance the stability of therapeutic agents that may be susceptible to chemical degradation by oxidation. Antioxidants are typically molecules that exhibit higher oxidative potential than the therapeutic agent or inhibit free radical-induced drug decomposition. Exemplary antioxidants include ascorbic acid derivatives (e.g., ascorbic acid, erythorbic acid, sodium ascorbate), thiol derivatives (e.g., thioglycerol, cysteine, acetylcysteine, cystine, dithioerytheritol, dithiothreitol, glutathione), buthylated hydroxyanisole (BHA), butylated hydroxytoluene (BHT), sulfurous acid salts (e.g., sodium sulfate, sodium bisulfite, acetone sodium bisulfite, sodium metabisulfite, sodium sulfite, sodium formaldehyde sulfoxylate, sodium thiosulfate), nordihydroguaiaretic acid, and vitamin E (e.g., tocopherols, tocotrienols). Vitamin E refers to a group of eight fat-soluble compounds that include both tocopherols and tocotrienols. Vitamin E has many biological functions; the antioxidant function being one of the most important and best known. It acts as such in the present compositions. Butylated hydroxytoluene (BHT) is a lipophilic organic compound, chemically a derivative of phenol. It acts as an antioxidant and antimicrobial compound in the present composition.

Chelating Agents

The use of chelating agents to stabilize chemicals and drugs in formulations is known. Chelating agents are scavengers for trace amounts of metal ions. Most commonly chelation involves a metal ion. Compounds which have this ability are known as chelating agents or chelating ligands. Many reactions, including many oxidation and decomposition reactions, are catalyzed by trace amounts of metallic ions present in formulations. Many drugs can be degraded through oxidation and hydrolytic reactions which are catalyzed by metal ions. The presence of metallic ions can therefore significantly accelerate the degradation of these drugs. Chelating agents are useful in preventing degradation for drugs in formulations. EDTA (ethylene diamine tetraacetic acid) and its salts are examples of powerful chelating agents. EDTA is known to stabilize drugs in solution by retarding their oxidation. Exemplary chelating agents include EDTA, a salt of EDTA (e.g., the disodium salt of EDTA), deferoxamine, sodium diethyldithiocarbamate, penicillamine, pentetate calcium, a sodium salt of pentetic acid, dimercaptosuccinic acid, triethylenetetramine, nitrilotriacetic acid, trans-diaminocyclohexanetetraacetic acid (DCTA), diethylenetriaminepentaacetic acid, bis(aminoethyl)glycolether-N,N,N′,N′-tetraacetic acid, iminodiacetic acid, acetic acid, tartaric acid, fumaric acid, or a salt thereof.

Edetate disodium is also known as the disodium salt of ethylenediaminetetraacetic acid (EDTA). EDTA is available in several salt forms, notably disodium EDTA and calcium disodium EDTA. EDTA is mainly used to sequester metal ions in aqueous solution. In personal care products, it is added to cosmetics to improve their stability toward air. It acts as a chelating agent that helps bind free radicals and impurities in the present composition.

pH Modifiers

There is often an optimum pH range for pharmaceutical formulations, both for compound stability and other factors such as skin permeation and solubility of therapeutic agents. This may require the pH to be adjusted during formulation due to the components of the formulation being too acidic or basic. Buffer systems are often used to maintain the pH within the desired range. Exemplary pH modifiers include acetic acid, adipic acid, ammonium carbonate, ammonium hydroxide, ammonium phosphate, boric acid, citric acid, diethanolamine, fumaric acid, hydrochloric acid, malic acid, nitric acid, propionic acid, potassium acetate, potassium bicarbonate, potassium chloride, potassium citrate, potassium metaphosphate, potassium phosphate, sodium acetate, sodium bicarbonate, sodium borate, sodium carbonate, sodium chloride, sodium citrate, sodium glycolate, sodium hydroxide, sodium lactate, sodium phosphate, sodium proprionate, succinic acid, sulfuric acid, tartaric acid, triethanolamine, or pharmaceutically acceptable salts thereof.

Triethanolamine is an organic compound that is both a tertiary amine and a triol. Like other amines, triethanolamine is a base and functions as a pH modifier in the present composition. Triethanolamine is used primarily as an emulsifier and surfactant. Triethanolamine neutralizes fatty acids, adjusts and buffers the pH, and solubilizes oils and other ingredients that are not fully soluble in water.

### Administration

The topical compositions can be administered in any number of ways. For example, the compositions in liquid form can be applied from absorbent pads; used to impregnate bandages and other dressings, or formulated as topical sprays. In another example, the composition in solid form, including semi-solid form, can be applied from a tube; or be applied directly onto the skin of the subject. In yet another example, the composition in liquid form or solid form can be applied by using an applicator to spread the composition onto the skin. The composition may also be applied to the skin under occlusive dressing in a dermal delivery system (e.g., a transdermal patch).

A composition is preferably applied to the skin as an ointment via an applicator containing at least one therapeutic agent on a swab applicator. Alternatively, the composition may be applied as a lotion, spray or foam from a tube or tin. For a topical spray, the compositions can include a clear aqueous ethanolic solution prepared by first combining the therapeutic agent or combinations of one or more therapeutic agents (e.g., a NSAID, such as, ketoprofen, ibuprofen, naproxen and salts thereof, pizoxicam, meloxicam, tenoxicam, mefenamic acid, meclofenamic acid, flufenamic acid, or celecoxib) in an amount in the range of about 1 to about 10 percent by weight, preferably about 5 percent by weight, based on the total weight of the solution, with lauryl lactate, lactic acid, and glyceryl monolaurate and dissolving the obtained admixture by gradual addition, at ambient temperature of alternating aliquots of water and ethanol, followed by the addition of propylene glycol. A non-ionic surfactant can also be used and can be added to the admixture prior to the addition of water or ethanol. In a further embodiment, the composition is applied as a lotion from a droptainer dispenser for larger volumes. In this embodiment, the composition may be applied using the fingers to rub the composition onto the skin. A transdermal patch with the composition or at least one therapeutic agent can also be used and applied.

The compositions and methods of the invention can include formulation(s) of compound(s) that, upon administration to a subject, result in a concentration of the compound(s) that treats peripheral neuropathy. The compound(s) may be contained in any appropriate amount in any suitable carrier substance, and are generally present in an amount of 2.5-15% by weight of the total weight of the composition. The composition may be provided in a dosage form that is suitable for the oral, parenteral (e.g., intravenously or intramuscularly), rectal, dermatological, cutaneous, nasal, vaginal, inhalant, skin (patch), ocular, intrathecal, or intracranial administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions according to the invention or used in the methods of the invention may be formulated to release the active compound immediately upon administration or at any predetermined time or time period after administration. The latter types of compositions are generally known as controlled release formulations, which include (i) formulations that create substantially constant concentrations of the agent(s) of the invention within the body over an extended period of time; (ii) formulations that after a predetermined lag time create substantially constant concentrations of the agent(s) of the invention within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per day; and (vi) formulations that target the action of the agent(s) by using carriers or chemical derivatives to deliver the combination to a particular target cell type. Administration of compound(s) in the form of a controlled release formulation is especially preferred for compounds having a narrow absorption window in the gastro-intestinal tract or a relatively short biological half-life.

Administration of therapeutic agents in controlled release formulations may be useful where the therapeutic agent has (i) a narrow therapeutic index (e.g., the difference between the plasma concentration leading to harmful side effects or toxic reactions and the plasma concentration leading to a therapeutic effect is small; (ii) a narrow slow absorption rate by or through the epithelium and/or dermis; or (iii) a short biological half-life, so that frequent dosing during a day is required in order to sustain a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound(s) are formulated with appropriate excipients into a pharmaceutical composition that, upon administration, releases the compound(s) in a controlled manner. Examples include single or multiple unit tablet or capsule compositions, oil solutions, suspensions, emulsions, microcapsules, molecular complexes, microspheres, nanoparticles, patches, and liposomes.

### EXAMPLES

The following examples are intended to illustrate rather than limit the invention.

### Example 1: Preparation of a Gel Base

Mixture A: In a suitable vessel, EDTA (5 g) was dissolved in purified water (5,500 g) with stirring. To the solution of EDTA in water, Carbopol 980 NF (50 g) was added with vigorous stirring until completely dissolved. Subsequently, Carbopol Ultrez (125 g) was added with vigorous stirring until completely dissolved. Mixture B: In a separate vessel, Cremophor (50 g), α-tocophenol acetate oil (5 g), ethyl alcohol (1 g), propylene glycol (1,000 g), isopropyl alcohol (900 g), isopropyl myristate (300 g), and benzyl alcohol (100 g) were mixed together with stirring. To this mixture, oxybenzone (500 g) and BHT (100 g) were added at 30-40° C. with stirring until completely dissolved. Mixture B was then added to mixture A with vigorous stirring. To the combined mixture, a solution of triethanolamine (100 g) in purified water (115 g) was added with vigorous stirring. A solution of triethanol amine (50 g) in purified water (100 g) was added until the pH was between 4.5-5.5 or purified water (100 g) was added if the pH was already between 4.5-5.5. The mixture was homogenized with a high viscosity mixer. This gel base was used to formulate the therapeutic agents into final formulations.

### Example 2: Preparation of a Gel Base without Benzyl Alcohol

Mixture A: In a suitable vessel, EDTA (5 g) was dissolved in purified water (5,500 g) with stirring. To the solution of EDTA in water. Carbopol 980 NF (50 g) was added with vigorous stirring until completely dissolved. Subsequently, Carbopol Ultrez (125 g) was added with vigorous stirring until completely dissolved. Mixture B: In a separate vessel, Cremophor (50 g), α-tocophenol acetate oil (5 g), ethyl alcohol (1 g), propylene glycol (1,000 g), isopropyl alcohol (900 g), and isopropyl myristate (300 g) were mixed together with stirring. To this mixture, oxybenzone (500 g) and BHT (100 g) were added at 30-40° C. with stirring until completely dissolved. Mixture B was then added to mixture A with vigorous stirring. To the combined mixture, a solution of triethanolamine (100 g) in purified water (115 g) was added with vigorous stirring. A solution of triethanol amine (50 g) in purified water (100 g) was added until the pH was between 4.5-5.5 or purified water (100 g) was added if the pH was already between 4.5-5.5. The mixture was homogenized with a high viscosity mixer. This gel base was used to formulate the therapeutic agents into final formulations.

### Example 3: Preparation of a Cream Base

To make 100 mL of a cream base, Carbomer NF, Isopropyl Myristate, Disodium EDTA, Sodium Methyl Paraben, and Sodium Propyl n Parabens were mixed using a suitable mixer as described in U.S. Pat. No. 6,083,996 to Buyuktimkin, et al. This cream base was used to formulate the therapeutic agents into final formulations.

### Example 4: Preparation of a Cream Base with DDAIP

To make 100 mL of a cream base, the hydrochloride or sulfuric acid salt of 2-dimethylaminopropionic acid dodecyl ester, Isopropyl Myristate, Disodium EDTA. Sodium Methyl Paraben, and Sodium Propyl n Parabens were mixed using a suitable mixer as described in U.S. Pat. No. 6,083,996 to Buyuktimkin, et al. This cream base was used to formulate the therapeutic agents into final formulations.

### Example 5: Preparation of Ointment with Therapeutic Agents

To prepare 50 g of drug product 0.5 g of micronized ketoprofen was dissolved in 15 g of ethyl alcohol (95%) and mixed with a formulation of Examples 1-4 (32.5 g) with vigorous mixing. To the resulting product triethanolamine (0.75 g) was added and the pH adjusted to between 5.3 and 5.5 under vigorous mixing.

### Example 6: Preparation of Topical Sprays with Therapeutic Agents

The topical compositions prepared in this example are clear, sprayable, aqueous ethanolic solutions including a nonsteroidal anti-inflammatory drug (NSAID). The aqueous spray solutions in this example contain the NSAID in the range of about 1 to about 10 percent by weight, preferably about 5 percent by weight. The aqueous spray solution also contains lauryl lactate (C₁₅H₃₀O₃) in the range of about 1 to about 5 percent by weight, lactic acid (C₃H₆O₃) in the range of about 0.5 to about 5 percent by weight, glyceryl monolaurate (C₁₅H₃O₄) in the range of about 2 to about 5 percent by weight. The aqueous spray solution optionally includes propylene glycol (C₃H₈O₂) in the range of about 5 to about 30 percent by weight. The remainder of the solution is constituted by water and ethanol in the range of about 0.3:1 to about 2.6:1 water:ethanol. The aqueous spray solution can also include a nonionic surfactant having a hydrophile-lipophile balance (HLB) value of at least 12. The nonionic surfactant can be polyethylene glycol ether of cetyl alcohol represented by the formula (CH₃(CH₂)₁₄CH₂(OCH₂CH₂)ₙOH, where n has an average of 10, and having a HLB value of about 15.7.

The aqueous spray solution is prepared by first combining the NSAID with lauryl lactate, lactic acid, and glyceryl monolaurate, and dissolving the mixture by gradual addition, at ambient temperature, of alternating aliquots of water and ethanol, followed by the addition of propylene glycol. The nonionic surfactant is added, if desired, to the mixture prior to the addition of water and ethanol.

The following tables provide the components by weight percentage of aqueous spray formulations with various therapeutic agents.

TABLE 1
Ketoprofen Spray Composition

| | Composition, wt. % | | | |
|---|---|---|---|---|
| Ingredients | KeS47 | KeS73 | KeS74 | KeS75 |
| Ketoprofen | 5 | 5 | 5 | 5 |
| Propylene glycol | 10 | 10 | 10 | 10 |
| Lauryl lactate | 3 | 3 | 3 | 3 |
| Lactic acid | 1.5 | 1.5 | 1.5 | 1.5 |
| Ceteth-20¹ | | 3 | | |
| Imwitor 412² | | | 3 | |
| Capmul PG-8³ | | | | 3 |
| Glyceryl monolaurate | 3 | 3 | 3 | 3 |
| Ethanol | 37.5 | 27.25 | 39.5 | 34.5 |
| Water | 40 | 47.25 | 35 | 40 |
| TOTAL | 100 | 100 | 100 | 100 |

¹CH₃(CH₂)₁₄CH₂(OCH₂CH₂)ₙOH, n average value 20; HLB 15.7; also Brij 58
²Propylene glycol laurate, HLB 4-5
³Propylene glycol monocaprylate, HLB 5-6

TABLE 2
Naproxen Spray Composition

| | Composition, wt. % | | | |
|---|---|---|---|---|
| Ingredients | NapS05 | NapS05a | NapS20 | Naprosyn ® |
| Naproxen | | | | 10 |
| Naproxen Na | 5 | 4.7 | 5 | |
| Propylene glycol | 10 | 9.5 | 10 | |
| Lauryl lactate | 3 | 2.8 | 3 | |
| Isopropyl myristate | | | 3 | |
| Lactic acid | 1.5 | 1.4 | 1.5 | |
| Ceteth-20¹ | | 2.8 | 3 | |
| Glyceryl monolaurate | 3 | 2.8 | 3 | |
| Ethanol | 55.5 | 42.7 | 41.5 | |
| Water | 22 | 33.2 | 30 | |
| TOTAL | 100 | 100 | 100 | |

TABLE 3
Ibuprofen Spray Composition

Composition, wt. %

| Ingredients | Ibu17 | Ibuleve ® |
|---|---|---|
| Ibuprofen | 5 | 5 |
| Lauryl lactate | 3 | |
| Lactic acid | 1.5 | |
| Glyceryl monolaurate | 3 | |
| Ethanol | 47.5 | |
| Water | 40 | |
| TOTAL | 100 | |

TABLE 4
Diclofenac Spray Composition

Composition, wt. %

| Ingredients | DcS02 | DcS03 | Swiss Relief ™ |
|---|---|---|---|
| Diclofenac Na | 1 | | 4¹ |
| Diclofenac diethylamine | | 1 | |
| Propylene glycol | 10 | 10 | |
| Lauryl lactate | 3 | 3 | |
| Lactic acid | 1.5 | 1.5 | |
| Glyceryl monolaurate | 3 | 3 | |
| Ethanol | 48.5 | 48.5 | |
| Water | 33 | 33 | |
| TOTAL | 100 | 100 | |

¹Swiss Relief ™ spray gel contains 4 wt. % diclofenac sodium together with inactive ingredients isopropyl alcohol, soy bean lecithin, ethanol, disodium phosphate, dodecahydrate, sodium dihodrogen phosphate dehydrate, sodium edetate, propylene glycol, peppermint oil, ascorbyl palmitate, hydrochloric acid (10% w/w), sodium hydroxide (10% w/w), and purified water.

TABLE 5
Ibuprofen Spray Composition

| | Composition, wt. % | | |
|---|---|---|---|
| Ingredients | Ibu17 | Ibu24 | Ibuleve ® |
| Ibuprofen | 5 | 5 | 5 |
| Propylene glycol | | 10 | |
| Lauryl lactate | 3 | 3 | |
| Lactic acid | 1.5 | 1.5 | |
| Glyceryl monolaurate | 3 | 3 | |
| Ethanol | 47.5 | 37.5 | |
| Water | 40 | 40 | |
| TOTAL | 100 | 100 | |

TABLE 6
Ketoprofen and a Nonionic Surfactant Spray Composition

| | Composition, wt. % | | | | |
|---|---|---|---|---|---|
| Ingredients | KeS47 | KeS61 | KeS67 | KeS73 | KeS73a |
| Ketoprofen | 5 | 5 | 5 | 5 | 5 |
| Propylene glycol | 10 | 10 | 10 | 10 | 10 |
| Lauryl lactate | 3 | 3 | 0 | 3 | 3 |
| Lactic acid | 1.5 | 1.5 | 1.5 | 1.5 | 1.5 |
| Brij 58 | | 3 | 3 | 3 | 3 |
| Glyceryl monolaurate | 3 | 0 | 3 | 3 | 3 |
| Ethanol | 37.5 | 37.5 | 37.5 | 27.25 | 25.25 |
| Water | 40 | 40 | 40 | 47.25 | 49.25 |
| TOTAL | 100 | 100 | 100 | 100 | 100 |

TABLE 7
Ketoprofen and Propylene Glycol Laurate Spray Composition

| | Composition, wt. % | | | |
|---|---|---|---|---|
| Ingredients | KeS47 | KeS62 | KeS68 | KeS74 |
| Ketoprofen | 5 | 5 | 5 | 5 |
| Propylene glycol | 10 | 10 | 10 | 10 |
| Lauryl lactate | 3 | 3 | 0 | 3 |
| Lactic acid | 1.5 | 1.5 | 1.5 | 1.5 |
| Propylene glycol laurate | | 3 | 3 | 3 |
| Glyceryl monolaurate | 3 | 0 | 3 | 3 |
| Ethanol | 37.5 | 42.5 | 42.5 | 39.5 |
| Water | 40 | 35 | 35 | 35 |
| TOTAL | 100 | 100 | 100 | 100 |

TABLE 8
Ketoprofen and Propylene Glycol Caprylate Spray Composition

| | Composition, wt. % | | | |
|---|---|---|---|---|
| Ingredients | KeS47 | KeS63 | KeS69 | KeS75 |
| Ketoprofen | 5 | 5 | 5 | 5 |
| Propylene glycol | 10 | 10 | 10 | 10 |
| Lauryl lactate | 3 | 3 | 0 | 3 |
| Lactic acid | 1.5 | 1.5 | 1.5 | 1.5 |
| Propylene glycol caprylate | | 3 | 3 | 3 |
| Glyceryl monolaurate | 3 | 0 | 3 | 3 |
| Ethanol | 37.5 | 37.5 | 37.5 | 34.5 |
| Water | 40 | 40 | 40 | 40 |
| TOTAL | 100 | 100 | 100 | 100 |

TABLE 9
Ketoprofen and Sorbitan Monolaurate Spray Composition

| | Composition, wt. % | | | |
|---|---|---|---|---|
| Ingredients | KeS47 | KeS66 | KeS72 | KeS78 |
| Ketoprofen | 5 | 5 | 5 | 5 |
| Propylene glycol | 10 | 10 | 10 | 10 |
| Lauryl lactate | 3 | 3 | 0 | 3 |
| Lactic acid | 1.5 | 1.5 | 1.5 | 1.5 |
| Sorbitan monolaurate | | 3 | 3 | 3 |
| Glyceryl monolaurate | 3 | 0 | 3 | 3 |
| Ethanol | 37.5 | 57.5 | 57.5 | 34.5 |
| Water | 40 | 20 | 20 | 40 |
| TOTAL | 100 | 100 | 100 | 100 |

TABLE 10
Ketoprofen and Nonionic Surfactant Spray Composition

| | Composition, wt. % | | | | | |
|---|---|---|---|---|---|---|
| Ingredients | KeS73 | KeS79 | KeS80 | KeS81 | KeS82 | KeS83 |
| Ketoprofen | 5 | 5 | 5 | 5 | 5 | 5 |
| Propylene | 10 | 10 | 10 | 10 | 10 | 10 |
| glycol | | | | | | |
| Lauryl lactate | 3 | 3 | 3 | 3 | 3 | 3 |
| Lactic acid | 1.5 | 1.5 | 1.5 | 1.5 | 1.5 | 1.5 |
| Brij 58 | 3 | | | | | |
| Brij 30 | | 3 | | | | |
| Brij 35 | | | 3 | | | |
| Brij 72 | | | | 3 | | |
| Brij 98 | | | | | 3 | |
| Brij 721 | | | | | | 3 |
| Glyceryl | 3 | 3 | 3 | 3 | 3 | 3 |
| monolaurate | | | | | | |
| Ethanol | 27.25 | 27.25 | 27.25 | 27.25 | 27.25 | 27.25 |
| Water | 47.25 | 47.25 | 47.25 | 47.25 | 47.25 | 47.25 |
| TOTAL | 100 | 100 | 100 | 100 | 100 | 100 |

TABLE 11
Ketoprofen Spray Composition

Composition, wt. %

| Ingredients | KeS38/25° C. | KeS38/40° C. |
|---|---|---|
| Ketoprofcn | 5 | 5 |
| Lauryl lactate | 3 | 3 |
| Lactic acid | 1.5 | 1.5 |
| Glyceryl monolaurate | 3 | 3 |
| Ethanol | 47.5 | 47.25 |
| Water | 40 | 40 |
| TOTAL | 100 | 100 |

TABLE 12
Naproxen Spray Composition Using 5% Naproxen
Sodium and Nonionic Surfactant

| | Composition, wt. % | | | | |
|---|---|---|---|---|---|
| Ingredients | NapS05 | NapS21a | NapS22a | NapS23a | NapS24 |
| Naproxen | 0 | 0 | 0 | 0 | 0 |
| Naproxen Na | 5 | 5 | 5 | 5 | 5 |
| Propylene glycol | 10 | 10 | 10 | 10 | 10 |
| Lauryl lactate | 3 | 3 | 3 | 3 | 3 |
| Lactic acid | 1.5 | 0.5 | 3 | 0.5 | 3 |
| Brij 58 | | 0 | 0 | 3 | 3 |
| Glyceryl | 3 | 3 | 3 | 3 | 3 |
| monolaurate | | | | | |
| Ethanol | 55.5 | 34.5 | 46 | 30 | 47 |
| Water | 22 | 44 | 30 | 45.5 | 26 |
| TOTAL | 100 | 100 | 100 | 100 | 100 |

TABLE 13
Naproxen Spray Composition Using 2.5% Naproxen
Sodium and a Nonionic Surfactant

| | Composition, wt. % | | | | | | |
|---|---|---|---|---|---|---|---|
| Ingredients | NapS05 | NapS25 | NapS25a | NapS26 | NapS26a | NapS27 | NapS28 |
| Naproxen | 0 | 0 | 0 | 0 | 0 | 0 | 0 |
| Naproxen Na | 5 | 2.5 | 2.5 | 2.5 | 2.5 | 2.5 | 2.5 |
| Propylene | 10 | 10 | 10 | 10 | 10 | 10 | 10 |
| glycol | | | | | | | |
| Lauryl lactate | 3 | 3 | 3 | 3 | 3 | 3 | 3 |
| Lactic acid | 1.5 | 0.5 | 0.5 | 1.5 | 1.5 | 0.5 | 1.5 |
| Brij 58 | | 3 | 3 | 3 | 3 | 0 | 0 |
| Glyceryl | 3 | 3 | 3 | 3 | 3 | 3 | 3 |
| monolaurate | | | | | | | |
| Ethanol | 55.5 | 22 | 32 | 37 | 40 | 36 | 45 |
| Water | 22 | 56 | 46 | 40 | 37 | 45 | 35 |
| TOTAL | 100 | 100 | 100 | 100 | 100 | 100 | 100 |

TABLE 14
Ibuprofen with and without Isopropyl
Myristate Spray Compositions

Composition, wt. %

| Ingredients | Ibu24 | Ibu30 |
|---|---|---|
| Ibuprofen | 5 | 5 |
| Propylene glycol | 10 | 10 |
| Isopropyl myristate | 0 | 3 |
| Lauryl lactate | 3 | 3 |
| Lactic acid | 1.5 | 1.5 |
| Glyceryl monolaurate | 3 | 3 |
| Ethanol | 37.5 | 39.5 |
| Water | 40 | 35 |
| TOTAL | 100 | 100 |

TABLE 15
Ibuprofen with Isopropyl Myristate
and Brij 58 Spray Compositions

| | Composition, wt. % | | |
|---|---|---|---|
| Ingredients | Ibu30 | Ibu32 | Ibu33 |
| Ibuprofen | 5 | 5 | 5 |
| Propylene glycol | 10 | 10 | 10 |
| Isopropyl myristate | 3 | 0 | 3 |
| Lauryl lactate | 3 | 3 | 3 |
| Lactic acid | 1.5 | 1.5 | 1.5 |
| Brij 58 | | 3 | 3 |
| Glyceryl monolaurate | 3 | 3 | 3 |
| Ethanol | 39.5 | 28 | 31.5 |
| Water | 35 | 46.5 | 40 |
| TOTAL | 100 | 100 | 100 |

TABLE 16
Diclofenac Spray Composition

| | Composition, wt. % | | | |
|---|---|---|---|---|
| Ingredients | DcS02 | DcS12 | DcS12a | DcS14 |
| Diclofenac Na | 1 | 1 | 1 | 1 |
| Propylene glycol | 10 | 15 | 10 | 10 |
| Lauryl lactate | 3 | 3 | 3 | 3 |
| Lactic acid | 1.5 | 1.5 | 1.5 | 0.5 |
| Brij 58 | | 3 | 3 | 3 |
| Glyceryl monolaurate | 3 | 3 | 3 | 3 |
| Ethanol | 48.5 | 25 | 43.5 | 34.5 |
| Water | 33 | 53.5 | 35 | 45 |
| TOTAL | 100 | 100 | 100 | 100 |

TABLE 17
Diclofenac Spray Composition

| | Composition, wt. % | | | | | |
|---|---|---|---|---|---|---|
| Ingredients | DcS03 | DcS13 | DcS13a | DcS13a-2 | DcS15 | DcS15a |
| Diclofenac | 1 | 1 | 1 | 1 | 1 | 1 |
| diethylamine | | | | | | |
| Propylene | 10 | 15 | 10 | 15 | 10 | 10 |
| glycol | | | | | | |
| Lauryl | 3 | 3 | 3 | 3 | 3 | 3 |
| lactate | | | | | | |
| Lactic acid | 1.5 | 1.5 | 1.5 | 1.5 | 0.5 | 0.5 |
| Brij 58 | 0 | 3 | 3 | 3 | 3 | 3 |
| Glyceryl | 3 | 3 | 3 | 3 | 3 | 3 |
| monolaurate | | | | | | |
| Ethanol | 48.5 | 23 | 38 | 45 | 26 | 39.5 |
| Water | 33 | 55.5 | 40.5 | 28.5 | 53.5 | 40 |
| TOTAL | 100 | 105 | 100 | 100 | 100 | 100 |

### Example 7: Pain Treatment in a Mouse Model

Mouse Model of PIPN—

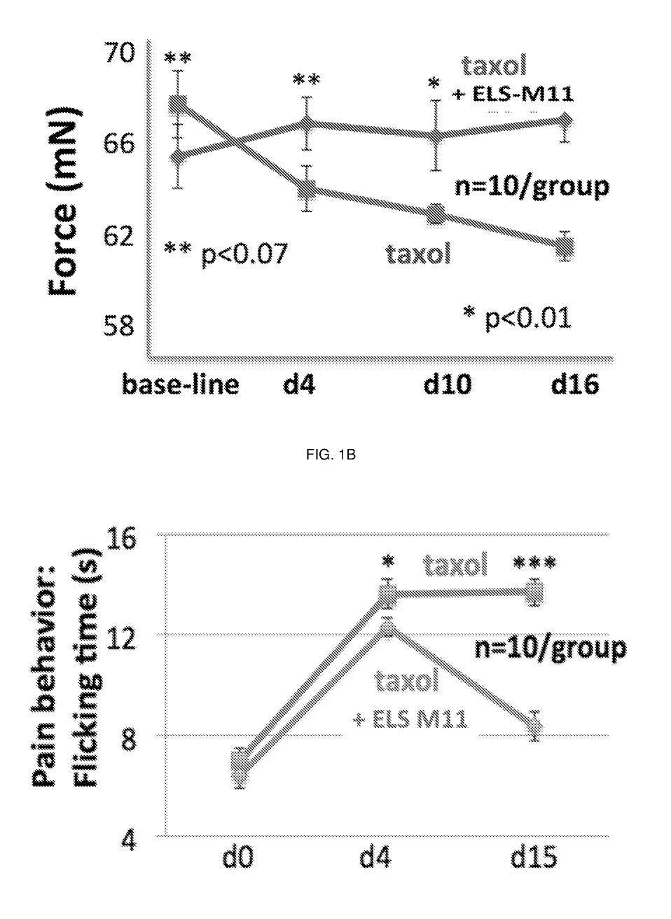

C57BL6j mice were exposed to paclitaxel (4 mg/kg bw) by 5 Injections on days 1, 3, 5, 7 and 9, following an established protocol for induction of clinically-relevant PIPN in mice REF. Mice were treated daily with topical-transdermal applications to hind-paws, starting day 1 until euthanasia on day 15. One group of mice were treated with ELS-M11, the control group with vehicle control. At the behavioral level, sensory withdrawal thresholds for mechanical cues, cold-induced no cifensive behavior, and conditional place preference—a pain-dependent choice behavior in freely moving animals—were measured on days 4 and 15. The results are illustrated in FIGS. 1A and 1B. ELS-M11 is disclosed in WO 2014/052313 and is registered under the trademark TOPOFEN™.

### Other Embodiments

All publications, patent applications, and patents mentioned in this specification are herein incorporated by reference in their entirety. Various modifications and variations of the described compositions, methods, and kits of the invention will be apparent to those skilled in the art without departing from the scope and spirit of the Invention. Although the Invention has been described in connection with specific embodiments, it will be understood that the invention as claimed should not be unduly limited to such specific embodiments. Indeed, various modifications of the described modes for carrying out the invention that are obvious to those skilled in the art are intended to be within the scope of the invention.

What is claimed is:

1. A method for treating peripheral neuropathy or for treating pain or a symptom associated with sunburn, said method comprising administering to a subject in need thereof an effective amount of a topical composition comprising:
a) a non-steroidal anti-inflammatory drug in the amount of 1 to 15 percent based on the weight of the composition;
b) a non-basic polymeric skin penetration enhancer present in an amount sufficient to enhance skin penetration of said non-steroidal anti-inflammatory drug, said polymeric skin penetration enhancer being a member of the group consisting of a water-dispersable acid polymer, a polysaccharide gum, or a mixture thereof; and a carrier consisting essentially of water;
c) a lipophilic solvent which is a mixture of one or more aliphatic C₂ to C₈ alcohols and an aliphatic C₈ to C₃₀ ester, said lipophilic solvent being present in an amount of about 10 percent to about 40 percent, wherein the ratio of the amount by weight of said aliphatic alcohol to the amount by weight of said ester is in the range of about 7 to about 1.

2. The method of claim 1, wherein said non-basic polymeric skin penetration enhancer is a cross-linked polyacrylic acid interpolymer, a cross-linked polyacrylic acid homopolymer, 2-dimethylaminopropionic acid dodecyl ester or a pharmaceutically acceptable salt thereof, or a mixture two or more thereof.

3. The method of claim 1 or 2, wherein said non-basic skin penetration enhancer is present in the amount of 1.5 to 1.75 percent based on the weight of the composition.

4. The method of any one of claims 1-3, wherein said lipophilic solvent is a mixture of ethyl alcohol, isopropyl alcohol, and isopropyl myristate.

5. The method of any one of claims 1-4, wherein said composition further comprises d) a chelating agent in the amount of 0.01 to 0.1 percent based on the weight of the composition.

6. The method of claim 5, wherein said chelating agent is the disodium salt of ethylenediamineteraacetic acid.

7. The method of any one of claims 1-6, wherein said composition further comprises e) oxybenzone in the amount of 1 to 7 percent based on the weight of the composition.

8. The method of any one of claims 1-7, wherein said composition further comprises f) an emulsifying agent in the amount of 0.01 to 1 percent based on the weight of the composition.

9. The method of claim 8, wherein said emulsifying agent is PEG-40 hydrogenated castor oil.

10. The method of any one of claims 1-9, wherein said composition further comprises g) a water-miscible alkylene glycol in the amount of 7 to 12 percent based on the weight of the composition.

11. The method of claim 10, wherein said water-miscible alkylene glycol is propylene glycol.

12. The method of any one of claims 1-11, wherein said composition further comprises h) a cosmetic preservative in the amount of 0.05 to 0.15 percent based on the weight of the composition.

13. The method of claim 12, wherein said cosmetic preservative is benzyl alcohol.

14. The method of any one of claims 1-3, wherein said composition further comprises i) an antioxidant or mixture of antioxidants in the amount of 1 to 1.1 percent based on the weight of the composition.

15. The method of claim 14, wherein said antioxidant or mixture of antioxidants is selected from Vitamin E, butylated hydroxytoluene, or a mixture thereof.

16. The method of any one of claims 1-15, wherein said composition further comprises j) a pH modifier in an amount sufficient to maintain a pH value of the composition in the range of 4.5 to 6.

17. The method of claim 16, wherein said pH modifier is triethanolamine.

18. The method of claim 1, wherein said topical composition comprises:
a) about 10 percent non-steroidal anti-inflammatory drug;
b) about 1.25 percent of a cross-linked polyacrylic acid interpolymer and about 0.5 percent of a cross-linked polyacrylic acid homopolymer;
c) about 19 percent of a mixture of one or more aliphatic alcohols and about 3 percent of an aliphatic ester;
d) about 0.05 percent of a chelating agent;
e) about 10 percent oxybenzone;
t) about 0.5 percent of an emulsifying agent;
g) about 10 percent of a water-miscible alkylene glycol;
h) about 1 percent of a cosmetic preservative;
i) about 1.05 percent of a mixture of antioxidants;
j about 1.5 percent of a pH modifier;
k) and the rest water.

19. The method of claim 1, wherein said topical composition comprises:
a) about 5 percent non-steroidal anti-inflammatory drug;
b) about 1.25 percent of a cross-linked polyacrylic acid interpolymer and about 0.5 percent of a cross-linked polyacrylic acid homopolymer;
c) about 19 percent of a mixture of one or more aliphatic alcohols and about 3 percent of an aliphatic ester;
d) about 0.05 percent of a chelating agent;
e) about 5 percent oxybenzone;
f) about 0.5 percent of an emulsifying agent;
g) about 10 percent of a water-miscible alkylene glycol;
h) about 1 percent of a cosmetic preservative;
i) about 1.05 percent of a mixture of antioxidants;
j) about 1.5 percent of a pH modifier;
k) and the rest water.

20. The method of claim 1, wherein said topical composition comprises:
a) about 2.5 percent non-steroidal anti-inflammatory drug;
b) about 1.25 percent of a cross-linked polyacrylic acid interpolymer and about 0.5 percent of a cross-linked polyacrylic acid homopolymer;
c) about 19 percent of a mixture of one or more aliphatic alcohols and about 3 percent of an aliphatic ester;
d) about 0.05 percent of a chelating agent;
e) about 3 percent oxybenzone;
f) about 0.5 percent of an emulsifying agent;
g) about 10 percent of a water-miscible alkylene glycol;
h) about 1 percent of a cosmetic preservative;
i) about 1.05 percent of a mixture of antioxidants;
j) about 1.5 percent of a pH modifier;
k) and the rest water.

21. The method of any one of claims 18-20, wherein said mixture of one or more aliphatic alcohols is a mixture of about 10 percent ethanol and about 9 percent isopropanol.

22. The method of any one of claims 18-21, wherein said aliphatic ester is isopropyl myristate.

23. The method of any one of claims 18-22, wherein said chelating agent is the disodium salt of ethylenediamineteraacetic acid.

24. The method of any one of claims 18-23, wherein said emulsifying agent is PEG-40 hydrogenated castor oil.

25. The method of any one of claims 18-24, wherein said water-miscible alkylene glycol is propylene glycol.

26. The method of any one of claims 18-25, wherein said cosmetic preservative is benzyl alcohol.

27. The method of any one of claims 18-26, wherein said mixture of antioxidants is a mixture of about 0.05 percent Vitamin E and about 1 percent butylated hydroxytoluene.

28. The method of any one of claims 18-27, wherein said pH modifier is triethanolamine.

29. The method of any one of claims 1-28, wherein said non-steroidal anti-inflammatory drug is selected from the list consisting of aspirin, diflunisal, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid, celecoxib, rofecoxib, valdecoxib, parecoxib, lumiracoxib, etoricoxib, firocoxib, paracetamol, nimesulide, licofelone, lysine clonixinate, and hyperforin, calcitriol.

30. The method of claim 29, wherein said non-steroidal anti-inflammatory drug is ketoprofen.

31. The method of any one of claims 1-30, wherein said composition further comprises gabapentin in the amount of 5 to 20 percent by weight of the composition.

32. The method of claim 31, wherein said composition comprises gabapentin in the amount of 8 to 10 percent by weight of the composition.

33. The method of any one of claims 1-32, wherein said topical composition is administered in combination with a neuroprotective agent, wherein said neuroprotective agent is administered systemically.

34. The method of claim 33, wherein said neuroprotective agent is selected from calcium, magnesium, glutathione, glutamine, carbamaepine, oxycarbazepine, vitamin E, erythropoietin, allopregnanolone, valproate, alpha-lipoic acid, acetyl-L-carnitine or a combination thereof.

35. The method of any one of claims 1-34, wherein said peripheral neuropathy is mononeuropathy, mononeuritis multiplex, polyneuropathy, autonomic neuropathy, or neuritis.

36. The method of any one of claims 1-35, wherein said peripheral neuropathy is chemotherapy induced peripheral neuropathy.

37. The method of any one of claims 1-35, wherein said peripheral neuropathy is diabetic neuropathy.

38. The method of any one of claims 1-32, wherein said symptom associated with sunburn is selected from the group consisting of: blisters, rashes, redness, swelling, tenderness, headaches, fevers, chills, or fatigue.

39. A method for treating peripheral neuropathy or for treating pain or a symptom associated with sunburn, said method comprising administering to a subject in need thereof an effective amount of a topical spray composition comprising:
a) a non-steroidal anti-inflammatory drug in the amount of about 1 to about 10 percent by weight of the composition;
b) lauryl lactate in the amount of about 1 to about 5 percent by weight;
c) lactic acid in an amount of about 0.5 to about 5 percent by weight;
d) glyceryl monolaurate in an amount of about 2 to about 5 percent by weight; and
e) a carrier consisting essentially of water and ethanol.

40. The method of claim 39, wherein said topical spray composition further comprises propylene glycol or a nonionic surfactant having an HLB value of at least 12.

41. The method of claim 40, wherein said nonionic surfactant is an alkoxylated alcohol.

42. The method of claim 41, wherein said alkoxylated alcohol is in an amount of about 0 to about 7 percent by weight.

43. The method of claim 40, wherein said propylene glycol is in an amount of about 5 to about 30 percent by weight.

44. The method of claim 39, wherein the water:ethanol weight ratio of said carrier is in the range of about 0.3:1 to about 2.6:1.

45. The method of claim 39, wherein said topical spray composition comprises:
a) about 5 percent by weight ketoprofen;
b) about 3 percent by weight lauryl lactate;
c) about 1.5 percent by weight lactic acid;
d) about 3 percent by weight glyceryl monolaurate;
e) about 3 percent by weight polyethylene glycol having a HLB value of about 15.7;
f) about 10 percent by weight propylene glycol; and
g) a water:ethanol weight ratio of about 1.7.

46. The method of any one of claims 39-44, wherein said non-steroidal anti-inflammatory drug is a propionic acid derivative selected from the group consisting of ketoprofen, ibuprofen, naproxen, and salts thereof; or an acetic acid derivative selected from the group consisting of diclofenac, indomethacin, etodolac, and salts thereof.

47. The method of claim 46, wherein said non-steroidal anti-inflammatory drug is ketoprofen.

48. The method of any one of claims 39-47, wherein said topical spray composition is administered in combination with a neuroprotective agent, wherein said neuroprotective agent is administered systemically.

49. The method of claim 48, wherein said neuroprotective agent is selected from calcium, magnesium, glutathione, glutamine, carbamaepine, oxycarbazepine, vitamin E, erythropoietin, allopregnanolone, valproate, alpha-lipoic acid, acetyl-L-carnitine or a combination thereof.

50. The method of any one of claims 39-49, wherein said peripheral neuropathy is mononeuropathy, mononeuritis multiplex, polyneuropathy, autonomic neuropathy, or neuritis.

51. The method of any one of claims 39-50, wherein said peripheral neuropathy is chemotherapy induced peripheral neuropathy.

52. The method of any one of claims 39-50, wherein said peripheral neuropathy is diabetic neuropathy.

53. The method of any one of claims 39-47, wherein said symptom associated with sunburn is selected from the group consisting of: blisters, rashes, redness, swelling, tenderness, headaches, fevers, chills, or fatigue.
